(19) United States
(12) Patent Application Publication
(10) Pub. No.: US 2010/0292535 A1
(43) Pub. Date: Nov. 18, 2010

(54) Endoscope with multiple fields of view

(76) Inventor: Larry Paskar, Chesterfield, MO (US)

Correspondence Address: Gregory E. Upchurch,;LegalMetric, 1000 Des Peres Road, Suite 210, St. Louis, MO, 63131, US

(21) Appl. No.: 12/454,458

(22) Filed: May 18, 2009

(51) Int. Cl. A61B 1/00

(52) U.S. Cl. 600/113

(57) ABSTRACT

An endoscope having first and second viewing elements provides separate views of the passage being traversed or the organ being inspected. The endoscope is particularly useful for traversing restrictions and forming desired shapes in situ.

### STATEMENT REGARDING FEDERALLY-SPONSORED RESEARCH/DEVELOPMENT

Not applicable.

### REFERENCE TO MICROFICHE APPENDIX

Not applicable.

### BACKGROUND OF THE INVENTION

This invention relates generally to endoscopic devices, and more particularly to such devices with multiple fields of view.

Endoscopes are used to assess surfaces of passages and/or organs in the human (or non-human) body. They conventionally include a tube for insertion into the body, a light delivery system to illuminate the organ or passage under inspection, an optical system for transmitting the image to the user, and an additional channel(s) to allow use of various medical instruments. The present invention is not limited to any particular endoscopic device, since it is well-suited for use with the vast variety of available endoscopes. Endoscopes are routinely used in visualizing the gastrointestinal tract (including the esophagus, stomach, duodenum, small intestine, colon, and bile duct), the respiratory tract, the urinary tract, vascular and other fluid channels, and various normally closed body cavities such as the abdominal cavity, joint interiors, thoracic cavity, and chest organs.

Although endoscopes are very helpful in all these applications, they could be improved. For example, during routine interrogation of the colon for endoscopic screening, areas beneath colonic folds may remain undetectable to diagnostic interrogation since sites inspected are forward to the scope view. In order to see rearward to the normal scope view, a very tight rearward-facing configuration of 180 degrees would have to be created by shaping the scope in a downgoing shape (the direction of withdrawal of the scope rather than its normal upgoing shape direction of insertion of the scope). This maneuver would require a great deal of time, and result in significant wear and tear on endoscopic elements (fibroptics, pull wires, etc.), particularly if the maneuver is repeated multiple times during a procedure.

It has been recently discovered that flat lesions in the colon are also more likely than previously thought to become cancerous, but are very difficult to detect using existing colonoscopes because they do not stand out in the forward-facing field of view of conventional colonoscopes. Moreover, existing endoscopes typically provide views in only one direction at a time, thereby giving an incomplete understanding of the surface or passage being inspected. In addition, conventional endoscopes suffer from difficulties in passing through restricted areas, areas with tight curvature of flexure, or other areas of tortuosity. At a minimum, this can resulted in failed procedures and sometimes can result in perforations of the passage by the endoscope.

In many instances, one could tell from the endoscope viewing element that a particular shape is needed to access a particular passage or organ, but there is no way other than the insertion of a separate catheter of the desired shape into the additional channel of the endoscope to access that passage. If the shape were not precisely the needed shape, that catheter would have to be removed from the endoscope and another catheter inserted until the passage or organ is successfully accessed.

### SUMMARY OF THE INVENTION

Among the various objects and features of the present invention may be noted the provision of an improved endoscope and method of using same with improved fields of view.

A second feature is the provision of an improved endoscope which allows the formation of a shape to be viewed by the user in situ, thereby facilitating the formation of the precise shape(s) needed to access desired passageways and organs.

A third feature is the provision of an improved endoscope which allows visualization of areas which previously could not be visualized using conventional endoscopes.

A fourth feature is the provision of an improved endoscope with improved ability to pass through tortuous or restricted passages in the body.

Briefly, in a first aspect of the present invention, an endoscope includes a main viewing element disposed in a tube sized to fit into a body, said main viewing element being disposed to provide a primary view, and a secondary viewing element disposed in said tube, said secondary viewing element being capable of being disposed so as to provide an auxiliary view which differs from the primary view.

In a second aspect of the present invention, a method of using an endoscope includes the steps of moving an endoscope having a main viewing element along a passage in the body until a restriction or tortuosity in said passage is reached, extending a secondary viewing element distally with respect to the main viewing element into the restriction or tortuosity, using the secondary viewing element to navigate the restriction or tortuosity, and moving the main viewing element of the endoscope distally along the path navigated by the secondary viewing element through the restriction and/or tortuosity.

In a third aspect of the present invention, a method of using an endoscope includes the steps of placing an endoscope having a main viewing element in a passage in a human body at a desired position, said main viewing element having a primary field of view, and using a secondary viewing element to image a portion of the passage outside the primary field of view, said secondary viewing element having an auxiliary field of view.

Other objects and features will be in part apparent and in part pointed out hereinafter.

### BRIEF DESCRIPTION OF THE DRAWINGS

FIG. 1 is a plan view of an endoscope of the present invention in a passage in a human body.

FIG. 2 is a view similar to FIG. 1 illustrating a different shape for the secondary viewing element of the endoscope.

FIG. 3 illustrates a down-going shape for the secondary viewing element.

FIG. 4 illustrates an up-going shape for the secondary viewing element.

FIG. 5 illustrates an out-of-plane shape for the secondary viewing element.

FIGS. 6, 7 and 7A-7C illustrate the use of the endoscope of the present invention in traversing a restriction or tortuosity in a passage in the human body.

FIG. 8 illustrates a second embodiment of the present invention in which the secondary viewing element is disposed out the side of the endoscope.

FIG. 9 illustrates the embodiment of FIG. 8 in which the secondary viewing element is further curved to view a proximal portion of the passage.

Similar reference characters indicate similar parts throughout the several views of the drawings.

### DESCRIPTION OF THE PREFERRED EMBODIMENT

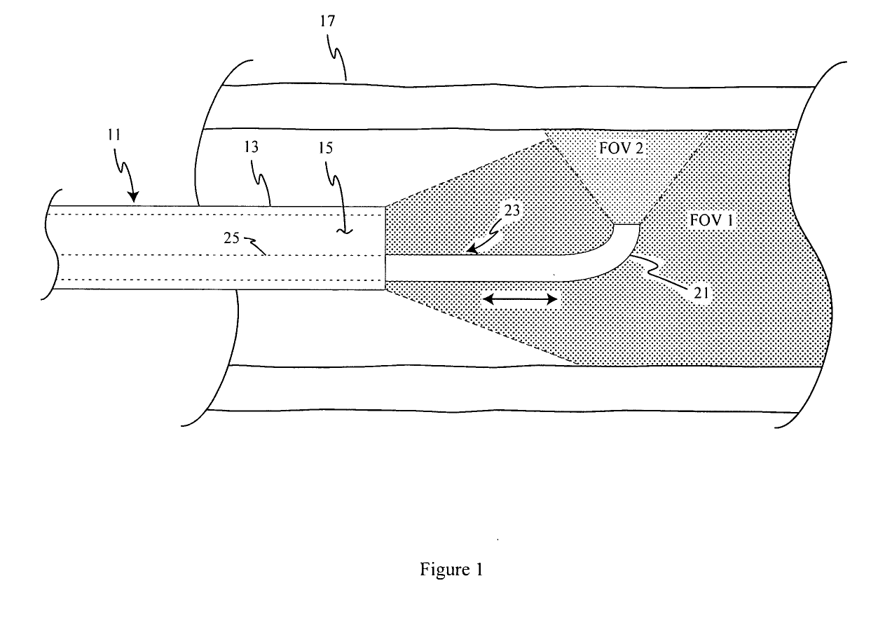

Turning to FIG. 1, an endoscope 11 of the present invention includes a tube 13 containing a conventional first viewing element 15 (such as a suitable fiber optic system for illuminating the primary field of view FOV1 and for transmitting the image of that field of view back to a user (not shown)). Of course, other systems using CMOS or CCD sensors or the like could also be used to acquire the image or images of the field of view (or views) of the endoscope of the present invention. Illumination could also be provided in any conventional manner. Tube 13 is sized to fit into a body such as the passage 17 shown in FIG. 1. It is anticipated that the invention may be used on human and non-human bodies. By way of illustration, passage 17 can be the colon of a human subject. An auxiliary viewing element 21 is included in a secondary tube 23 which is movable in the additional channel 25 of endoscope 11. Secondary tube 23 is preferably curved or curvable and may consist of multiple parts, as described below. The field of view FOV2 of secondary viewing element 21 may (depending upon the position of secondary tube 23 with respect to endoscope tube 13) differ from the primary view FOV1. In FIG. 1, the fields of view FOV1 and FOV2 overlap, but the field of view FOV1 is primarily forward-looking with respect to the endoscope 11, while the field of view FOV2 is primarily lateral-looking.

Secondary tube 23, along with secondary viewing element 21 which it carries, may be extended or retracted longitudinally with respect to the first viewing element 15 as indicated by the double-pointed arrow in FIG. 1.

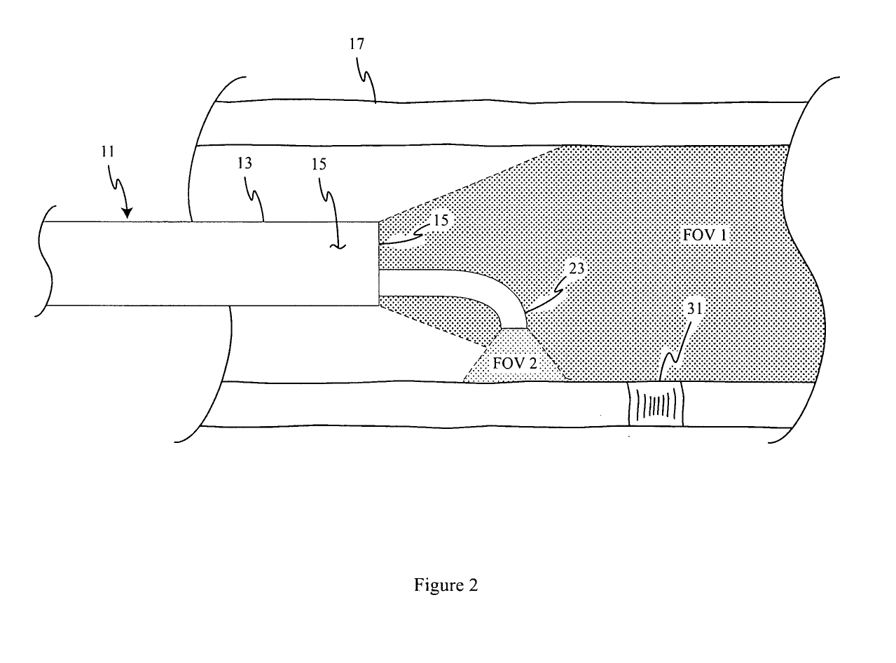

As will become apparent below, the distal section of secondary tube 23 may be shaped by the user to a vast variety of shapes while the distal section is disposed in the passage 17. In FIG. 2, the secondary tube 23 has been reshaped from the shape of FIG. 1 to that of FIG. 2 while being in the field of view FOV1 of the primary viewing element 15. That is, using the configuration of FIGS. 1 and 2, the actual shape of the secondary tube 23 may be viewed as that shape is changed. Such a change may be desirable, for example, to access orifice 31 in passage 17.

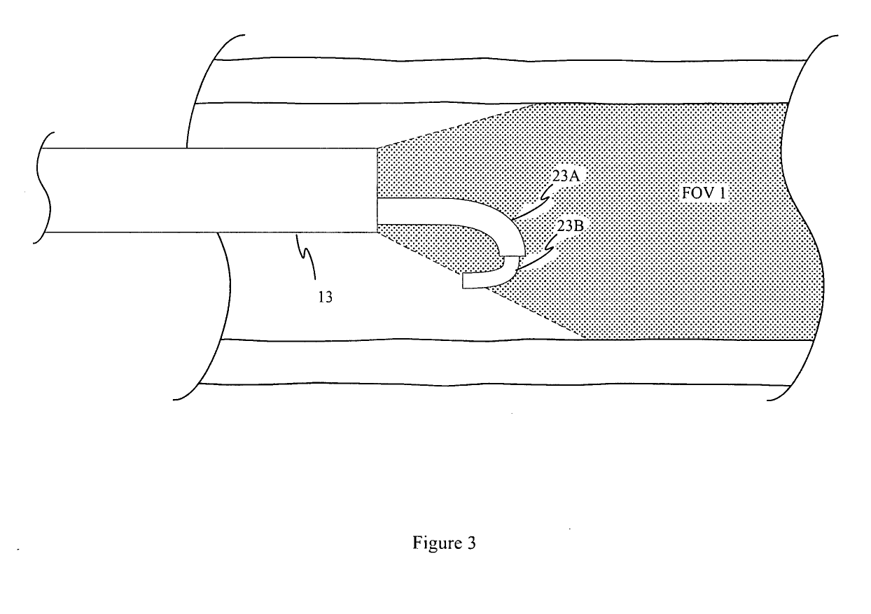

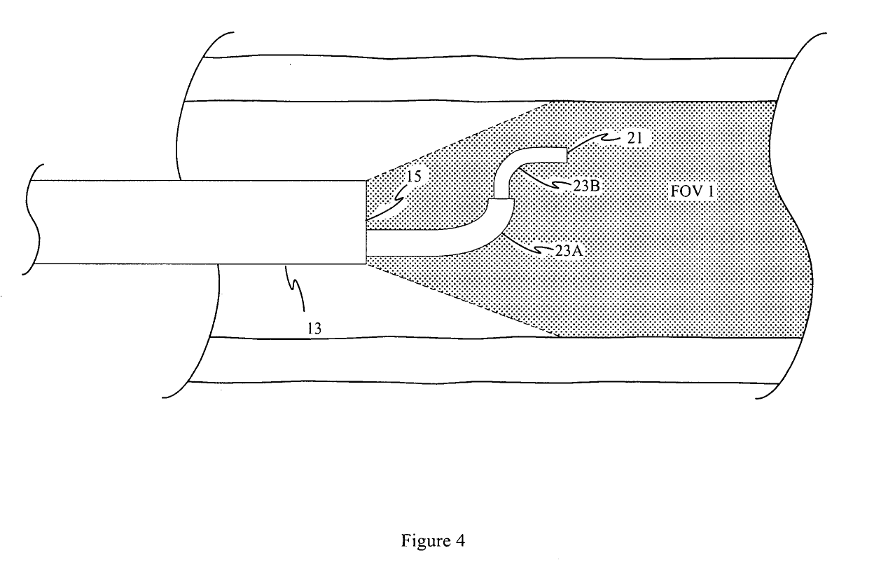

The simple shape change from FIG. 1 to FIG. 2 may be accomplished by rotating the secondary tube 23 with respect to primary tube 13 and/or by use of a conventional pull-wire mechanism. But it is preferred that secondary tube 23 be shapeable in other ways. To provide maximum shapeability, secondary tube 23 preferably (see FIG. 3) is a composite structure composed of two independently controllable tubes (23A and 23B), both of which are capable of assuming a curved shape. When both tubes 23A and 23B are curved in the same direction, extreme curvature of composite tube 23 may be achieved as shown in FIG. 3. That Figure illustrates a down-going shape for the composite tube (the distal end of the tube faces in a direction opposed to the direction the tube as a whole would move were it inserted farther into passage 17). Similarly, in FIG. 4, a complex up-going shape (the distal end of the tube faces in the direction in which the tube as a whole would move were it inserted farther into passage 17) has been created in the field of view of element 15. This shape, and a multitude of similar shapes, may be formed by rotating tube 23A with respect to tube 23B 180 degrees from the position of FIG. 3 and bending the distal portions of each tube a desired amount by the use of pullwires (not shown) or the like.

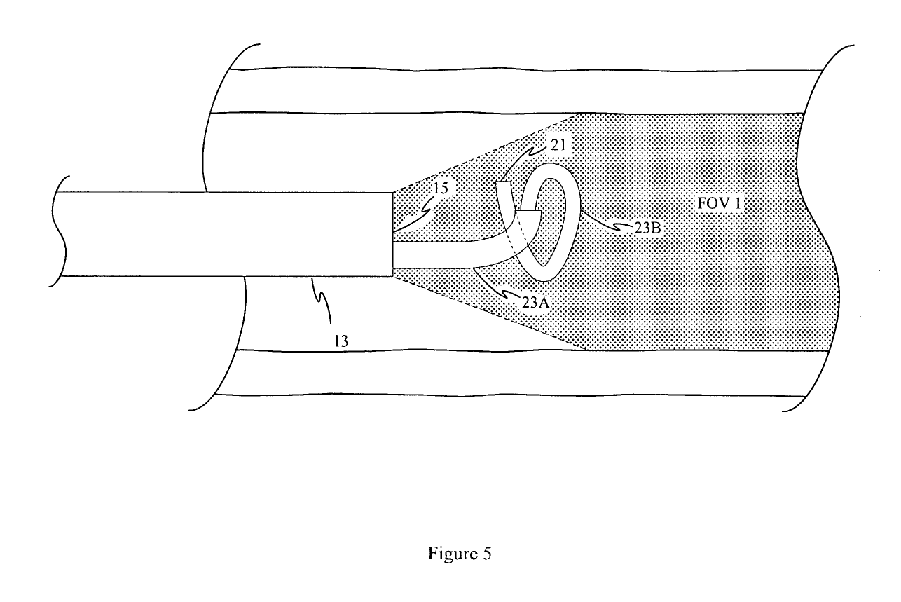

It should be understood (see FIG. 5) that the secondary tube 23 may be formed into shapes which are other than simple up-going and/or down-going shapes. By rotating the inner secondary tube 23B with respect to outer tube 23A and allowing the curved portions of both tubes to interact, a composite shape of the distal portion of composite tube 23 is formed which is out-of-plane (in this case perpendicular) to the longitudinal axis of primary tube 13. Such out-of-plane shapes are known, but heretofore are not believed to have been available for endoscopes. More particularly, it is not believed that such shapes have heretofore been made under visual inspection by the user in situ.

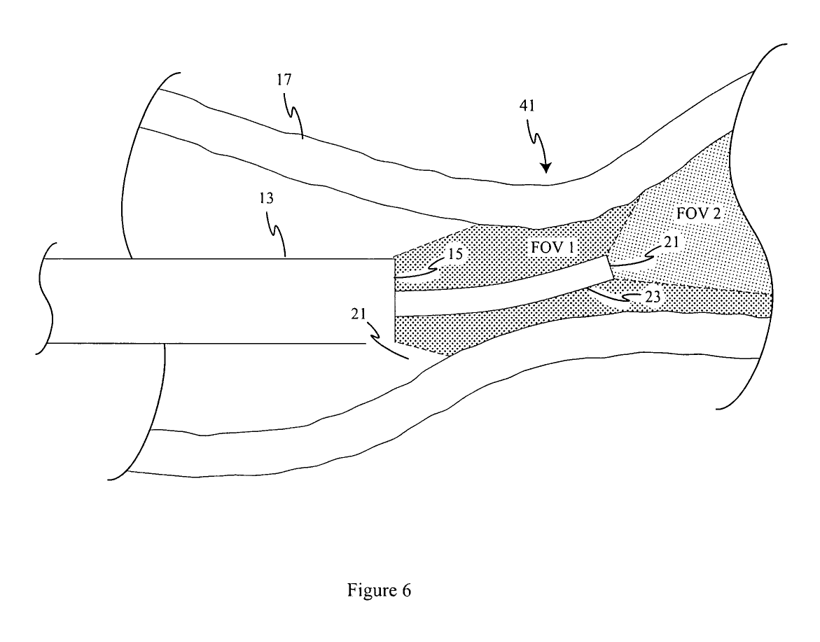

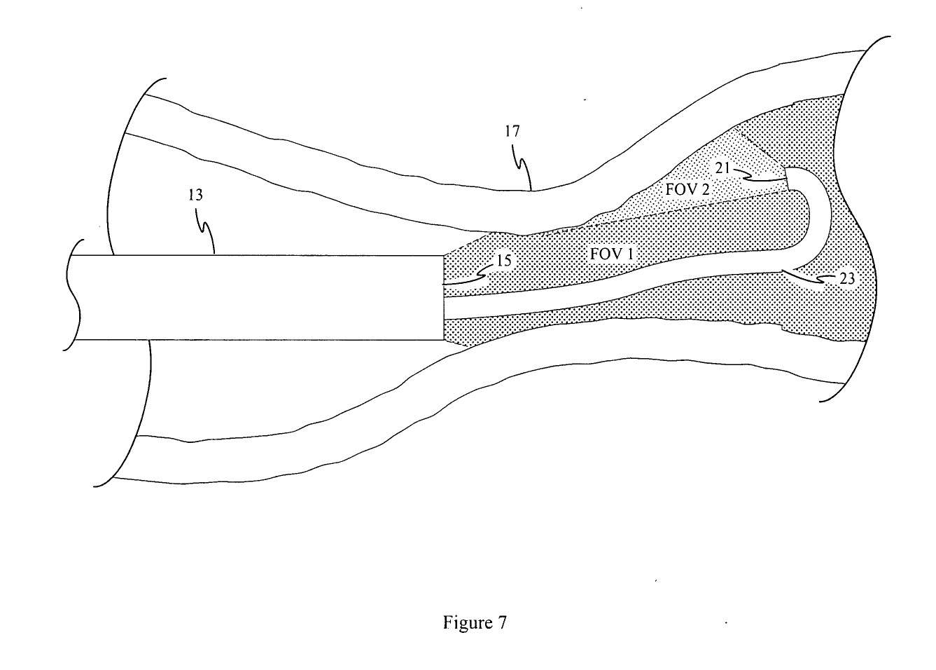

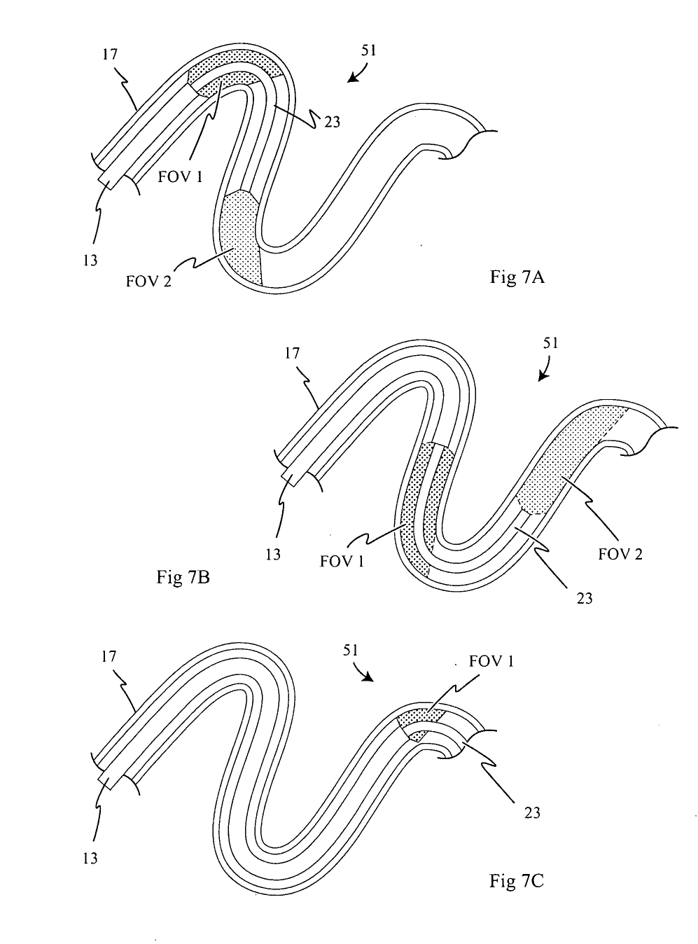

The endoscope 11 of the present invention is particularly well-suited to traversing restrictions in passage 17 (see FIGS. 6 and 7). In FIG. 6, the restriction 41 is seen in the field of view FOV1 of the primary viewing element. Secondary tube 23 is extended through the restriction, guided by the image from secondary viewing element 21 while the restriction is being traversed. Once the secondary tube 23 successfully passes through the restriction, it may be shaped into a down-going curve as discussed above so that the field of view FOV2 of the secondary tube now includes the distal side of the restriction. The primary tube 13 is then advanced over secondary tube 23 while the secondary viewing element is held fixed with respect to the passage to safely traverse the restriction while the process is being imaged by both viewing elements.

Endoscope 11 is also well-suited for traversing tortuosity in passage 17 (see FIGS. 7A-7C). Although the tortuosity 51 is shown in two-dimensions in FIGS. 7A-7C, it should be realized that the tortuosity is routinely in three-dimensions, which makes passage therethrough even more difficult than that illustrated in FIGS. 7A-7C. The sigmoid tortuosity 51 shown in passage 17 can be successfully imaged and traversed by endoscope 11 as follows: As outer tube 13 approaches the first curve of the tortuosity, inner tube 23 is curved into the “clockwise” curve shown in FIG. 7A and extended around the first curve. Note that the field of view FOV1 of the outer tube 13 is such that the shape into which inner tube 23 is formed can be visually verified to be appropriate to the curve to be traversed. The shape of inner tube 23 may be adjusted as the curve of the tortuosity changes since the distal portion of the curve is visually available to the user since it falls in the field of view FOV2 of the inner tube 23. After the inner tube 23 has been advanced through the curve a distance sufficient to provide the necessary purchase for endoscope 11, outer tube 13 is advanced over inner tube 23 to the position indicated in FIG. 7B. At that point, the second curve of tortuosity 51 is encountered, so the process is repeated. Specifically, outer tube 13 may be used to view the tortuosity 51 and the inner tube 23 in field of view FOV1 to determine both the appropriate curvature of inner tube 23 and whether tube 23 actually assumes the appropriate shape. Inner tube 23 is curved into the “counterclockwise” curve illustrated in FIG. 7B (which is accomplished by rotating the inner element 180 degrees with respect to the outer tube and then curving the inner tube). Curves are referred to as clockwise and counterclockwise herein with reference to the view shown in FIG. 7A. If viewed from the opposite direction, the “clockwise” curve would become “counterclockwise” and vice versa, but from all points of view the curves are opposite each other in direction of curvature. Once inner tube 23 has been advanced a distance through the second curve to achieve the necessary purchase, outer tube 13 is then advanced over inner tube 23 to the position shown in FIG. 7C. The third curve can then be traversed by recurving inner tube 23 into the clockwise curved shape shown in FIG. 7C. Specifically, in FIG. 7C the outer tube 13 may again be used to view the tortuosity 51 and the inner tube 23 in field of view FOV1 to determine both the appropriate curvature of inner tube 23 and whether tube 23 actually assumes the appropriate shape. The process can be repeated as needed to overcome any type of tortuosity. If the necessary shape for the inner tube to traverse a particular tortuosity is out-of-plane with respect to the distal end portion of the outer tube 13, the inner tube 23 can be formed into the required out-of-plane shape by rotating the inner tube with respect to the outer tube by some required angle other than 180 degrees. Of course, inner tube 23 can also (if it is composed of two separate curved or curvable elements) be formed into an out-of-plane shape as described above in connection with FIG. 5 and that formation can be observed by element 13 so long as it occurs in field of view FOV1.

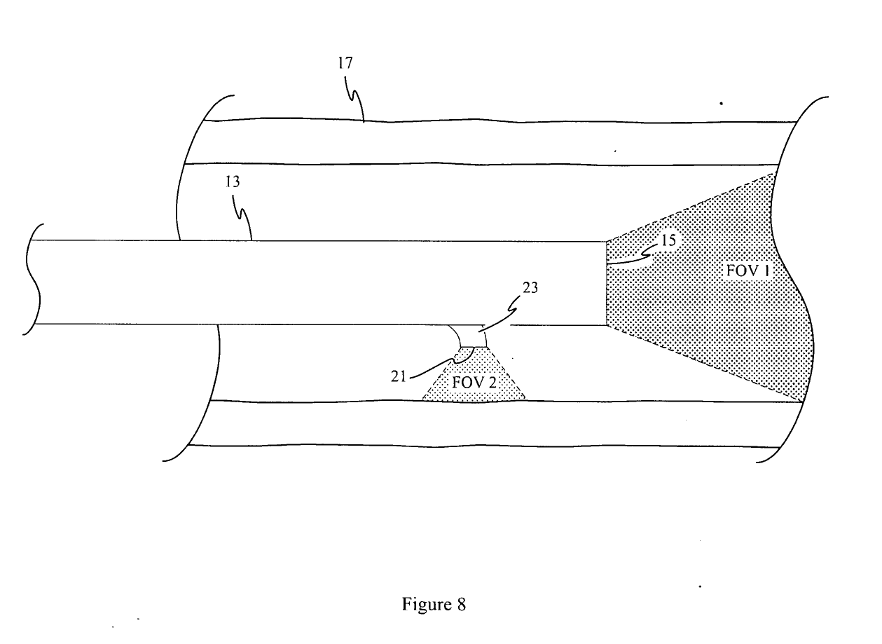

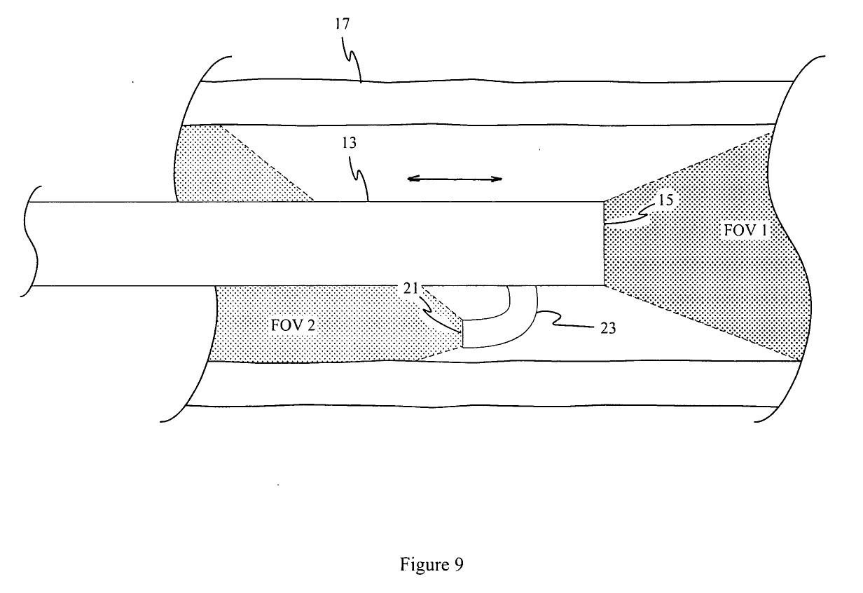

Although FIGS. 1-7C illustrate secondary tube 23 being disposed in the field of view FOV1 of the primary viewing element 15, the present invention is not so limited. In FIGS. 8 and 9, the secondary tube 23 with secondary viewing element 21 exits the side of primary tube 13 so that the side of the passage (FIG. 8) or the proximal portion of the passage (FIG. 9) may be visually imaged while the primary viewing element is imaging the distal portion of the passage. It should be understood that varying the amount of curvature of tube 23 as described above, changes the field of view FOV2 from that of FIG. 8 to that of FIG. 9. Any desired curvature may be imposed upon secondary tube 23 to obtain the desired secondary field of view FOV2. As indicated in FIG. 9 by the double-arrow, both proximal and distal portions of the passage may be imaged both as the endoscope 11 is being inserted and as it is being removed.

In view of the above it will be seen that the various objects and features of the present invention are achieved and other advantageous results obtained.

What is claimed is:

1. An endoscope comprising:
a main viewing element disposed in a tube sized to fit into a body, said main viewing element being disposed to provide a primary view;
a secondary viewing element disposed in said tube, said secondary viewing element being capable of being disposed so as to provide an auxiliary view which differs from the primary view, said secondary viewing element having an auxiliary field of view which is controllably positionable to a plurality of positions with respect to the primary field of view.

2. The endoscope as set forth in claim 1 wherein the secondary viewing element is extendible with respect to the main viewing element.

3. The endoscope as set forth in claim 2 wherein the secondary viewing element is extendible distally with respect to the main viewing element.

4. The endoscope as set forth in claim 3 wherein the secondary viewing element is extendible distally into the primary view of the main viewing element so that the secondary. viewing element when so extended is viewed by the main viewing element.

5. The endoscope as set forth in claim 4 wherein the secondary viewing element is shapeable, said secondary viewing element being extendible distally an amount sufficient to allow the shape of the secondary viewing element to be viewed by the main viewing element as said shape is being formed.

6. The endoscope as set forth in claim 2 wherein the secondary viewing element is extendible transversely from the main viewing element.

7. The endoscope as set forth in claim 1 wherein the secondary viewing element is formable into a down-going shape with respect to the tube.

8. The endoscope as set forth in claim 1 wherein the secondary viewing element is formable into an out-of-plane shape with respect to the tube.

9. The endoscope as set forth in claim 1 wherein the secondary viewing element is formable into an up-going shape with respect to the tube.

10. The endoscope as set forth in claim 1 wherein the secondary viewing element is formable into a rearward pointing shape with respect to the tube.

11. A method of using an endoscope comprising:
moving an endoscope having a main viewing element along a passage in a body until a restriction or tortuosity in said passage is reached;
extending a secondary viewing element distally with respect to the main viewing element into the restriction or tortuosity, using the secondary viewing element to navigate the restriction;
moving the main viewing element of the endoscope distally along the path navigated by the secondary viewing element through the restriction or tortuosity.

12. The method as set forth in claim 11 further including forming the secondary viewing element into a shape to allow viewing the restriction by the secondary viewing element once the secondary viewing element has passed through the restriction.

13. The method as set forth in claim 11 wherein the secondary viewing element is smaller than the main viewing element.

14. The method as set forth in claim 11 wherein the secondary viewing element is shapeable, said secondary viewing element being formed into a desired shape to navigate the restriction.

15. The method as set forth in claim 11 wherein the main viewing element is moved distally to navigate the restriction while the secondary viewing element is held fixed with respect to the passage.

16. A method of using an endoscope comprising:
placing an endoscope having a main viewing element in a passage in a body at a desired position, said main viewing element having a primary field of view;
using a secondary viewing element to image a portion of the passage outside the primary field of view, said secondary viewing element having an auxiliary field of view which is controllably positionable to a plurality of positions with respect to the primary field of view.

17. The method as set forth in claim 16 further including the step of disposing the secondary viewing element so that the auxiliary field of view is oriented approximately 90 degrees with respect to the primary field of view.

18. The method as set forth in claim 16 further including the step of disposing the secondary viewing element so that the auxiliary field of view is oriented approximately 180 degrees with respect to the primary field of view.

19. The method as set forth in claim 16 further including the step of disposing the secondary viewing element so that the auxiliary field of view and the primary field of view overlap.

20. The method as set forth in claim 16 further including disposing the secondary viewing element to face distally.

21. The method as set forth in claim 20 wherein the endoscope is at least partially withdrawn from the passage while the secondary viewing element is facing rearwardly.

22. The method as set forth in claim 16 wherein the distal end portion of the main viewing element defines a plane, further including disposing the secondary viewing element out of said plane.

23. The method as set forth in claim 16 wherein said secondary viewing element is extended distally and the shape of the secondary viewing element is viewed by the main viewing element as said shape is being formed.
